(19) United States
(12) Patent Application Publication
(10) Pub. No.: US 2009/0082451 A1
(43) Pub. Date: Mar. 26, 2009

(54) USE OF THYROID HORMONE CONVERSION INHIBITORS

(75) Inventors: Joshua D. Safer, Chestnut Hill, MA (US); Michael F. Holick, Sudbury, MA (US)

Correspondence Address: KNOBBE MARTENS OLSON & BEAR LLP, 2040 MAIN STREET, FOURTEENTH FLOOR, IRVINE, CA, 92614, US

(73) Assignee: Trustees of Boston University, Boston, MA (US)

(21) Appl. No.: 11/661,479

(22) PCT Filed: Aug. 30, 2005
(86) PCT No.: PCT/US2005/030919

§ 371 Date: Dec. 8, 2008

Related U.S. Application Data
(60) Provisional application No. 60/606,481, filed on Sep. 1, 2004.

(51) Int. Cl. A61K 31/195; A61K 31/138; A61P 17/00

(52) U.S. Cl. 514/567; 514/652

(57) ABSTRACT

The present invention is directed to the use of thyroid hormone conversion inhibitors to treat hyperproliferative skin disorders, preferably their use in topical admixtures.

The present application claims benefit under 35 U.S.C. §119(e) of provisional application U.S. Ser. No. 60/606,481, filed Sep. 1, 2004.

### FIELD OF THE INVENTION

The present invention is directed to the use of thyroid hormone conversion inhibitors to treat hyperproliferative skin disorders, preferably their use in topical admixtures.

### BACKGROUND OF THE INVENTION

A number of diseases are associated with hyperproliferation of cells, including psoriasis, the ichthyoses, cancer and cutaneous viral infections. For example, psoriasis is a chronic inflammatory disease characterized by hyperproliferation and impaired differentiation of keratinocytes.

Psoriasis is one of the most common dermatologic diseases, affecting up to 1 to 2 percent of the world's population. It is characterized by erythematous, sharply demarcated papules and rounded plaques, covered by silvery micaceous scale. The skin lesions of psoriasis are variably pruritic. Traumatized areas often develop lesions of psoriasis. Additionally, other external factors may exacerbate psoriasis including infections, stress, and medications, e.g. lithium, beta blockers, and anti-malarials.

The most common variety of psoriasis is called plaque type. Patients with plaque-type psoriasis will have stable, slowly growing plaques, which remain basically unchanged for long periods of time. The most common areas for plaque psoriasis to occur are the elbows, knees, gluteal cleft, and the scalp. Involvement tends to be symmetrical. Inverse psoriasis affects the intertriginous regions including the axilla, groin, submammary region, and navel, and it also tends to affect the scalp, palms, and soles. The individual lesions are sharply demarcated plaques but may be moist due to their location. Plaque-type psoriasis generally develops slowly and runs an indolent course. It rarely spontaneously remits.

Eruptive psoriasis (guttate psoriasis) is most common in children and young adults. It develops acutely in individuals previously without psoriasis or in those with chronic plaque psoriasis. Patients have many small erythematous, scaling papules, frequently after upper respiratory tract infection with beta-hemolytic streptococci. Patients with psoriasis may also develop pustular lesions. These may be localized to the palms and soles or may be generalized and associated with fever, malaise, diarrhea, and arthralgias.

The existing treatments for psoriasis are targeted at the major histopathologic components of the disease. Psoriasis is caused by unknown factors that stimulate T-lymphocyte activation, proliferation, and cytokine release that leads to hyperproliferation of keratinocytes that overproduce Bcl-x (instead of normal Bcl-2) and therefore resist apoptosis. Cells that mediate the skin manifestations of psoriasis reside primarily in the epidermis or at the dermal-epidermal interphase. Although a few CD4⁺ T-cells may be present in lesional skin, the majority of the cells are CD8⁺ lymphocytes that secrete cytokines such as interleukin-2 and interferon-gamma. These cytokines drive proliferation of keratinocytes and endothelial cells of the microvessels in affected skin. The keratinocytes in psoriatic lesions neither differentiate normally into compact and protective stratum corneum, nor are these cells subject to apoptosis like normal keratinocytes. This is because the psoriatic keratinocytes, due to the effect of IFN-gamma, contain Bcl-x. Bcl-x protects against Fas-mediated apoptotic proteins. Normal cells that contain Bcl-2 are susceptible to Fas-mediated apoptosis.

A variety of different approaches have been taken to target the major histopathologic components of the disease. For example, broad immunosuppression or T-lymphocyte specific immunosuppression is achieved by treatment with UVB, cyclosporine, methotrexate, topical steroids, and other immunosuppressive modalities. Keratinocyte terminal differentiation is targeted by calcipotriene and salicyclic acid. Retinoids target both immunosuppression and keratinocyte terminal differentiation.

Topical treatments have been used as an adjunct to other therapies in patients with moderate to severe psoriasis. In individuals with limited to moderate psoriasis, such typical treatments by themselves may be sufficient. The typical topical therapies include coal tar preparation (1-5% by weight). Although this is the most frequently used topical therapy, coal tar has a bad odor and stains clothing. Coal tar is thought to be effective for psoriasis because it is toxic to T cells, but is not toxic to skin cells. However, as discussed above, it has only limited utility by itself in individuals with moderate or higher states of psoriasis.

Another type of topical therapy includes steroids, but long term use of fluorinated corticostetoids (which are more effective than hydrocortisone) can lead to striae, telangiectasis and ecchythmosis. Topical anthralin cream (1%) or high dose/short duration anthralin in 1% salicylic acid in petroleum may be effective, or the topical synthetic retinoid tazarotene, may also provide short-term relief. However, these ingredients are often irritating and can cause other undesired side effects.

Another type of topical therapy is the use of retinoids, for example, in U.S. Pat. Nos. 3,934,028; 3,966,967; 4,021,573 and 4,216,224. However, retinoids can also provide undesired side effects, particularly in women of child-bearing age. Other topical agents such as calcipotriene, a vitamin D analogue (vitamin D3 or calcipotriol) may also provide temporary relief, while keratolytics such as salicylic acid can help in removing the thick scales from the psoriatic plaques. See, for example, U.S. Pat. No. 4,483,845 “Systemic Treatment of Psoriasis Using Certain Salicylates.”

Another type of topical therapy includes thioureylenes and thiabendazole, for example in U.S. Pat. No. 5,310,742 and U.S. published Patent Application No. 2004/0116387. This class of antithyroid drugs, including for example propylthiouracil (PTU) and methimazole (MMI), has effects on thyroid hormone biosynthesis, exhibits immunomodulatory effects, and functions in scavenging free radicals. PTU has been used to treat patients with psoriasis based on its immunomodulatory effects, for example, decreased production of IgM and IgG, decreased activity of immunoglobulin-secreting cells in plaque forming assays, augmentation of NK cell activity, increased percentage of total and suppressor/cytotoxic cells and reduced activated lymphocytes. Recently, it has been suggested that the utility of PTU in topical treatments of psoriasis may be due to antiproliferative effects, by a mechanism involving retinoic X receptor heterodimer formation with other receptors of the steroid receptor superfamily, including the retinoic acid receptor and vitamin D receptor. (Elias, Med. Hypotheses 62:431-7 (2004)). However, these agents are associated with significant side effects, including agranulocytosis, and the development of a mild, sometimes purpuric, popular rash. The use of additional thyroid hormone receptor agonists, as well as antagonists, to treat psoriasis has also been described. For example, U.S. Patent Application 20040116387 describes the use of receptor agonists, while U.S. Patent Applications 20040097589, 20040039028, and 20030166724 describe the use of receptor agonists to treat disorders including psoriasis.

Despite their limited effectiveness and the side effects associated with their use, topical therapies are the mainstay of treatment for moderate psoriasis, while they are used as adjunctive therapy in patients with more severe disease. The number of different and sometimes toxic treatments employed for amelioration of psoriasis is testimony to the resistant nature of this disease. Not only is moderate to severe psoriasis relatively resistant to topical treatments, but because of its chronic and recurrent nature, systemic therapy or radiation is often required. The devastating nature of this disease is emphasized by the extent of the side effects that psoriasis sufferers are willing to endure to attain a remission to a disease that they know will recur sooner or later.

Accordingly, there remains a need for improved treatments for psoriasis.

### SUMMARY OF THE INVENTION

We have surprisingly discovered that thyroid hormone conversion inhibitors, such as deiodinase inhibitors, inhibit epidermal proliferation. Accordingly, the present invention provides compositions and methods for topically inhibiting the conversion of thyroid pro-hormone, T₄, to thyroid hormone, T₃, including for the treatment of hyperproliferative skin disorders.

In one embodiment, the invention provides a topical composition containing as its active ingredient a thyroid hormone conversion inhibitor for the treatment of hyperproliferative skin disorders. In one embodiment the inhibitor is a deiodinase inhibitor. More specifically, the invention provides compositions and methods for a pharmaceutical composition comprising a thyroid hormone conversion inhibitor. Preferably, the thyroid hormone conversion inhibitor is a deiodinase inhibitor such as an iodinated contrast agent, e.g., iopanoic acid (IOP) and/or its analogs.

The invention provides a pharmaceutical composition comprising an inhibitor of the conversion of thyroid pro-hormone, T₄, to thyroid hormone, T₃, and a pharmaceutically acceptable carrier or diluent. One preferred thyroid hormone conversion inhibitor is iopanoic acid. Preferably, the carrier or diluent is for topical administration, including liposome creams.

The pharmaceutical compositions of the invention are useful for topical treatment of a hyperproliferative skin disorder. One preferred hyperproliferative skin disorder is psoriasis. Other hyperproliferative skin disorders include acne vulgaris, acne rosacea, actinic keratosis, solar keratoses, squamous carcinoma in situ, the ichthyoses, hyperkeratoses, disorders of keratinization such as Darriers disease, palmoplanter keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus and lichen planus.psoriasis, acne vulgaris, or cancer.

Preferably, the topical administration of the compositions of the present invention allows local lowering of the level of thyroid hormone but does not significantly alter the systemic levels of thyroid hormone in the subject.

### BRIEF DESCRIPTION OF THE DRAWINGS

FIG. 1 is a schematic of the experimental design for analyzing the effects of iopanoic acid administered topically to mice in vivo.

FIG. 2 is a graph which shows that epidermal proliferation was significantly diminished in Group 1 mice, treated with a control vehicle first followed by treatment with IOP.

FIG. 3 is a graph which shows that epidermal proliferation was significantly diminished in Group 2 mice, treated with IOP first followed by treatment with a control vehicle.

FIG. 4 is a graph which shows the epidermal thickness for control and IOP treated mice.

FIG. 5 is a graph which shows that relative to control cultures, proliferation of human epidermal keratinocytes was inhibited 61±9% (p<0.001) after incubation overnight with iopanoic acid.

FIG. 6 is a graph which shows that proliferation of human epidermal keratinocytes was inhibited 14±6% (p<0.01) after incubation overnight with ipodate sodium.

FIG. 7 is a graph which shows that proliferation of keratinocytes was inhibited 62±3% (p<0.001) after overnight incubation with propranolol.

FIG. 8 is graph which shows that relative to control cultures, proliferation of human prostate cells was inhibited 26±4% (p<0.001) after incubation overnight with iopanoic acid.

FIG. 9 is a graph which shows that proliferation of human prostate cells was inhibited 30±5% (p<0.001) after incubation overnight with ipodate sodium.

FIG. 10 is a graph which shows that proliferation of prostate cells was inhibited 84±8% (p<0.001) after overnight incubation with propranolol.

FIG. 11 is a graph which shows that relative to control cultures, proliferation of opossum kidney cells was inhibited 88±10% (p<0.001) after overnight incubation with propranolol.

FIG. 12 is a graph which shows that proliferation of monkey kidney cells was inhibited 82±5% (p<0.001) after overnight incubation with propranolol.

FIG. 13 is a graph which shows the results of the human epidermal keratinocytes incubated with two different concentrations of iopanoic acid.

FIG. 14 is a graph which shows the results of the human epidermal keratinocytes incubated with two different concentrations of ipodate sodium.

FIG. 15 is a graph which shows the results of the human epidermal keratinocytes incubated with two different concentrations of propanolol.

FIG. 16 is a graph which shows the results of the human prostate cells incubated with two different concentrations of iopanoic acid.

FIG. 17 is a graph which shows the results of the human prostate cells incubated with two different concentrations of ipodate sodium.

FIG. 18 is a graph which shows the results of the human prostate cells incubated with two different concentrations of propanolol.

In FIG. 19, proliferation of human prostate cells incubated overnight with ioversol was inhibited 22% (+/−7%, p<0.001).

### DETAILED DESCRIPTION OF THE INVENTION

We have surprisingly discovered that thyroid hormone conversion inhibitors, such as deiodinase inhibitors, inhibit epidermal proliferation. Accordingly, the present invention provides compositions and methods for topically inhibiting the conversion of thyroid pro-hormone, T₄, to thyroid hormone, T₃, including for the treatment of hyperproliferative skin disorders.

The extensive role of thyroid hormones in regulating metabolism in humans is well recognized. Thyroid hormones affect the metabolism of virtually every cell of the body. At normal levels, these hormones maintain body weight, the metabolic rate, body temperature, and mood, and influence serum low density lipoprotein (LDL) levels. In hypothyroidism there is weight gain, high levels of LDL cholesterol, and depression. In hyperthyroidism, these hormones lead to weight loss, hypermetabolism, lowering of serum LDL levels, cardiac arrhythmias, heart failure, muscle weakness, bone loss in postmenopausal women, and anxiety.

The predominant circulating thyroid hormone is the pro-hormone, T₄. Active thyroid hormone is generated by the conversion of thyroid pro-hormone, T₄, into thyroid hormone, T₃. Many individual tissues express their own thyroid deiodinases and depend on local T₄ conversion to the active thyroid hormone, T₃. We have previously discovered that epidermal growth depends on thyroid hormone and that topical administration of T₃ can markedly stimulate local epidermal proliferation.

We have now discovered that inhibiting the conversion of thyroid pro-hormone, T₄, into thyroid hormone, T₃, inhibits proliferation of epidermal cells. Accordingly, the present invention provides compositions and methods for the use of thyroid hormone conversion inhibitors, such as deiodinases, e.g., iopanoic acid (IOP). One embodiment provides topical administration of a thyroid hormone conversion inhibitor for the treatment of hyperproliferative skin disorders, including psoriasis.

Thyroid hormone conversion inhibitors of the invention preferably include agents other than propyl thiouracil (PTU) and, glucocorticoids that inhibit the conversion of thyroid pro-hormone, T₄, into thyroid hormone, T₃. The ability of a compound to inhibit the conversion of thyroid pro-hormone to thyroid hormone can readily be determined in vitro using standard assays. Certain compounds have different effects on inhibition at different concentrations.

Thyroid hormone conversion inhibitors are well known in the art. Examples of thyroid hormone conversion inhibitors include but are not limited to iodine containing contrast agents such as iopanoic acid and ipodate, amiodarone, glucocorticoids such as hydrocortisone and dexamethasone, propyl thiouracil, and propanolol, and their analogs. It is preferred that the inhibitor is not PTU or a glucocorticoid.

In one preferred embodiment, the thyroid hormone conversion inhibitor is an agent used as a iodinated contrast medium. Iodinated contrast media of the invention are sometimes referred to as iodinated contrast agents or contrast agents or cholegraphic media. Iododinated contrast agents include but are not limited to water-soluble, hepatotrophic contrast media; water-soluble, nephrotropic high osmolar contrast media; water-soluble, nephrotropic low osmolar contrast media; and non-water soluble contrast media. In one preferred embodiment the agent is a water-soluble, hepatotrophic X-ray contrast medium. Water-soluble hepatrotophic media include but are not limited to Iodoxamic acid; Iotroxic acid; Ioglycamic acid; Adipiodone; Iobenzamic acid; Iopanoic acid; Iocetamic acid; Sodium iopodate; Tyropanoic acid; and Calcium iopodate. Water-soluble, nephrotropic, high osmolar X-ray contrast media include but are not limited to Diatrizoic acid; Metrizoic acid; Iodamide; Iotalamic acid; Ioxitalamic acid; Ioglicic acid; Acetrizoic acid; Iocarmic acid; Methiodal; and Diodone. Water-soluble, nephrotropic, low osmolar X-ray contrast media include but are not limited to Metrizamide; Iohexyl; Ioxaglic acid; Iopamidol; Iopromide; Iotrolan; Ioversol; Iopentol; Iodixanol; Iomeprol; Iobitridol; and Ioxilan. Non-watersoluble X-ray contrast media include but are not limited to ethyl esters of iodised fatty acids; Iopydol; Propyliodone; and Iofendylate.

Particularly preferred contrast agents are iopanoic acid (also commonly known as its tradenames including Telepaque, Cistobil, Colegraf, Felombrine, and Jopanonsyre) and ipodate (also known as orgrafin). Sodium ipodate is administered to adults as a contrast agent at 0.5-3 grams/day.

In one preferred embodiment, the thyroid hormone conversion inhibitor is amiodarone.

In one preferred embodiment, the thyroid hormone conversion inhibitor is propanolol. High doses of propranolol, greater than 160 mg/day, inhibit the conversion of T4 to T3. Esmolol is another ultra-short acting beta blocking agent that can be used in the methods of the invention. Other beta blocking agents that can be used in the methods of the invention include but are not limited to guanethidine and reserpine.

Any amount of the agent that will inhibit conversion of T4 to T3 when applied topically can be used in the present invention. 0.1-40% of the agent is used, in certain embodiments at less 1% of the agent is used, one can use at least 5% of the agent, in other embodiments, at least 10%. However, various concentrations can be used such as 5-30%, 10-25%, 10-20% and all ranges from 1 to 40%.

### Hyperproliferative Disorders

The compositions of the present invention are useful for the treatment of any disorder of the epidermis characterized by hyperproliferation. Preferably, the compositions and methods of the invention inhibit proliferation and enhance differentiation of skin cells.

The methods and compositions of the invention are useful for treatment of a variety of hyperproliferative diseases. In particular, the methods and compositions of the invention are especially useful for the treatment of psoriasis. It will be appreciated however that cells of patients suffering from skin hyperproliferative diseases (as described in further detail below) as well as certain cancer cells such as prostate cancer cells may share many properties with each other.

In general, the methods and compositions of the invention may be used to treat or alleviate the symptoms of a patient suffering from any disease in which there is an imbalance between proliferation and differentiation. For example, any condition in which there is a failure in the normal controls which regulate the differentiative or proliferative fate of the cell may be treated. Such a disease will typically involve a cell or tissue type proliferating which normally (i.e., depending on the developmental stage or tissue type) does not or should not proliferate, or which fails to differentiate when the corresponding normal cell or tissue type is in a differentiated state. In a particular embodiment, the methods and compositions are suitable for treating, etc, a hyperproliferative disease, in particular a hyperproliferative disease which affects the skin. Neoplasms and cancer are also suitably treated, and other diseases and conditions are disclosed below.

“Antiproliferative” is used herein to denote effects on the skin including, but not limited to decreasing inflammation, to retarding or normalizing epidermal proliferation and keratinization to produce beneficial effects on hyperproliferative disorders of the skin.

The methods of the invention result in a reduction of proliferation, preferably proliferation in vivo, of the hyperproliferative cells. More preferably, proliferation of a population of cells is reduced to 90%, 80% 70%, 60%, 50%, 40%, 30%, 20%, or less compared to a similar population of untreated cells. Most preferably, proliferation is reduced to 0%, i.e., the cells cease dividing completely.

As used here, the term “proliferation” is intended to mean the division of cells resulting in growth of a tissue. Proliferative cells are actively dividing, and undergo such cell cycle processes as DNA replication, mitosis, cell division etc. Various methods are known by which proliferation may be assayed, for example, by radiolabelling with radioactive nucleotide triphosphates, tritiated thymidine, bromodeoxyuridine etc to detect replicating cells, by visual examination for mitotic cells etc. Proliferation may also assayed by expression of markers such as Ki-67, or by determining the increase in cell numbers by direct counting of cultured cells under different conditions. A preferred method of assaying reduction of proliferation is by measurement of mitotic index. “Mitotic index” as used here means the percentage of cells in a given population which are undergoing mitosis and/or cell division. Other assays are possible, for example, measurement of cell cycle period.

As used here, the term “hyperproliferation” means increased proliferation compared to expected proliferation for a cell type, given its stage of development and function.

In a highly preferred embodiment of the invention, the methods result in cell differentiation occurring within some or all of the population of treated cells. Preferably, 10% or more of a hyperproliferative cell population undergoes differentiation after treatment according to the invention compared with a population of untreated cells. More preferably, this percentage is 20%, 30%, 40%, 50%, 60%, 70%, 80% or more. Most preferably, 90%, 95% or 100% of the cell population undergoes differentiation.

“Differentiation” refers to the process by which unspecialized cells of tissues become specialized for particular functions. Differentiation of a cell may be assessed in various ways, for example morphologically, or by assaying expression of protein markers specific for the differentiated cell type as known in the art. For example, K1 and K10 keratin are markers for commitment to terminal differentiation of epidermal keratinocytes, and expression is increased when cellular differentiation occurs. In addition to K1 and K10 keratin, other keratin subtypes may be used as markers for different differentiation stages, for example, K5, K14, K16 and K17. Other non-keratin markers, for example EGF-receptor and .beta.-1 integrin, may also be used as markers for cellular differentiation.

Hyperproliferative skin disorders of the present invention are sometimes referred to as skin proliferation diseases, disorders, or conditions, or as skin proliferative diseases, disorders, or condition, or as hyperproliferative diseases, disorders, or conditions.

Hyperproliferative skin disorders which may be treated by using the methods and compositions of the present invention include psoriasis and its varied clinical forms, acne vulgaris, acne rosacea, actinic keratosis (solar keratoses—squamous carcinoma in situ), the ichthyoses, hyperkeratoses, disorders of keratinization such as Darriers disease, palmoplanter keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus, lichen planus, Reiter's syndrome, and hyperproliferative variants of the disorders of keratinization.

While not wishing to be bound by theory, the methods and composition of the present invention are also useful for the treatment of a variety of diseases and condition associated with activation of the androgen receptor, including but not limited to prostate carcinoma, benign prostate hyperplasia, alopecia, acne, oily skin, and hirsutism. The methods and composition of the present invention are also useful for the treatment of wound healing by decelerating the speed of wound healing to enhance quality of the scar.

According to the invention, a patient exhibiting any of the symptoms associated with a skin hyperproliferative disease, for example, a disease as listed above, can be treated with a thyroid hormone conversion inhibitor, such as IOP. Such treatment leads to reduced proliferation of the diseased cells. The inhibitor may be applied to a patient on its own, on in the form of a pharmaceutical composition as described in more detail below. The effect of treatment of a host with a skin proliferation disease may be evaluated by objective criteria such as an improvement of desquamation and erythema, reduction of the size of lesions as well as subjective criteria such as cessation of itching.

In one particularly preferred embodiment, the compositions and methods of the present invention are suitable for the treatment or alleviation of symptoms of psoriasis. Psoriasis manifests itself as inflamed swollen skin lesions covered with silvery white scale. Characteristics of psoriasis include pus-like blisters (pustular psoriasis), severe sloughing of the skin (erythrodermic psoriasis), drop-like dots (guttate psoriasis) and smooth inflamed lesions (inverse psoriasis).

The causes of psoriasis are currently unknown, although it has been established as an autoimmune skin disorder with a genetic component. One in three people report a family history of psoriasis, but there is no pattern of inheritance. However, there are many cases in which children with no apparent family history of the disease will develop psoriasis. Whether a person actually develops psoriasis may depend on “trigger factors” which include systemic infections such as strep throat, injury to the skin (the Koebner phenomenon), vaccinations, certain medications, and intramuscular injections or oral steroid medications. Once something triggers a person's genetic tendency to develop psoriasis, it is thought that in turn, the immune system triggers the excessive skin cell reproduction.

Psoriasis is a genetically determined disease of the skin characterized by two biological hallmarks. First, there is a profound epidermal hyperproliferation related to accelerated and incomplete differentiation. Second, there is a marked inflammation of both epidermis and dermis with an increased recruitment of T lymphocytes, and in some cases, formation of neutrophil microabcesses. Many pathologic features of psoriasis can be attributed to alterations in the growth and maturation of epidermal keratinocytes, with increased proliferation of epidermal cells, occurring within 0.2 mm of the skin's surface. Traditional investigations into the pathogenesis of psoriasis have focused on the increased proliferation and hyperplasia of the epidermis. In normal skin, the time for a cell to move from the basal layer through the granular layer is 4 to 5 weeks. In psoriatic lesions, the time is decreased sevenfold to tenfold because of a shortened cell cycle time, an increase in the absolute number of cells capable of proliferating, and an increased proportion of cells that are actually dividing. The hyperproliferative phenomenon is also expressed, although to a substantially smaller degree, in the clinically uninvolved skin of psoriatic patients.

A common form of psoriasis, psoriasis vulgaris, is characterized by well-demarcated erythematous plaques covered by thick, silvery scales. A characteristic finding is the isomorphic response (Koebner phenomenon), in which new psoriatic lesions arise at sites of cutaneous trauma.

Lesions are often localized to the extensor surfaces of the extremities, and the nails and scalp are also commonly involved. Much less common forms include guttate psoriasis, a form of the disease that often erupts following streptococcal pharyngitis, and pustular psoriasis, which is characterized by numerous sterile pustules, often 2 to 5 mm in diameter, on the palms and soles or distributed over the body.

Objective methods which are employed for establishing the effect of treatment of psoriasis patients include the resolution of plaques by visual monitoring and with photography. The visual scoring is done using PASI (Psoriasis Area and Severity Index) score (see Fredericksson, A J, Peterssonn B C Dermatologies 157: 38-244 (1978)).

The methods and compositions of the present invention are also suitable for the treatment of acne. Acne affects large patient populations and is a common inflammatory skin disorder which usually localizes on the face. Fortunately, the disease usually disappears and in the interval of months or years between onset and resolution, therapy, although not curative, can satisfactorily suppress the disease in the majority of patients.

A small number of acne patients with severe disease show little or no response to intensive therapeutic efforts including the use of high doses of oral tetracycline, dapsone, prednisone, and, in women, estrogen. In many cases, these drugs afford only a modest degree of control while the side effects of these agents severely restrict their usefulness. Patients with nodulocystic acne suffer from large, inflammatory, suppurative nodules appearing on the face, and frequently the back and chest. In addition to their appearance, the lesions are tender and often purulently exudative and hemorrhagic. Disfiguring scars are frequently inevitable. Therapies for acne involve local and systemic administration of retinoids. Topical application of all-trans-retinoic acid (tretinoin) has been tried with some success, particularly against comedones or blackheads, but this condition frequently returns when the treatment is withdrawn.

In another preferred embodiment, the methods and compositions of the invention may be used for inhibiting the proliferation and optionally reversing the transformed phenotype of hyperproliferative cancer. In particular, the methods and compositions described are useful for treating any tumor, carcinoma, lesion, etc, which is characterized by hyperproliferation of the skin. The methods and compositions are also useful to treat pre-malignant conditions i.e. to prevent their progression to actual malignancy, and to prevent the spread of tumors. Specific examples of tumors include melanocytic naevus and mrelodysplastic syndrome. Yet other examples include reduction of the number and/or prevention of progression of pre-cancerous actinic keratoses and bowenoid keratoses, treatment of established squamous cell carcinomas, and resolution of dysplastic naevi.

### Compositions

For therapeutic applications, a thyroid hormone conversion inhibitor may be suitably administered to a patient, alone or as part of a pharmaceutical composition, comprising the thyroid hormone conversion inhibitor together with one or more acceptable carriers thereof and optionally other therapeutic ingredients. The carrier(s) must be “acceptable” in the sense of being compatible with the other ingredients of the formulation and not deleterious to the recipient thereof.

As used herein a thyroid hormone conversion inhibitor of the invention is an agent that inhibits the conversion of thyroid pro-hormone, T₄, into thyroid hormone, T₃. Preferably the thyroid hormone conversion inhibitor is a deiodinase. Even more preferably, the thyroid hormone conversion inhibitor is iopanoic acid (IOP) or propranolol.

In one particularly preferred embodiment of the invention, a locally administrable topical pharmaceutical composition is provided for the treatment of a skin hyperproliferative disorder. The locally administrable topical composition includes a topical carrier. The compositions of the present invention include those suitable for topical and systemic administration including oral, rectal, intravaginal, nasal, ophthalmic or parenteral administration, all of which may be used as routes of administration using the materials of the present invention. A preferred route of administration is topical. The topical composition may be in the form of a pharmaceutical but it does not have to be. For example, it can be a cosmetic.

The formulations may conveniently be presented in unit dosage form, e.g., liposomes, tablets and sustained release capsules, and may be prepared by any methods well know in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liposomes, liquid carriers, or finely divided solid carriers or both, and then if necessary shaping the product.

Preferably, the compositions of the invention are encapsulated within liposomes. Liposomes suitable for use in the invention can be formed from standard vesicle-forming lipids, which generally include neutral or negatively charged phospholipids and a sterol such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9:467; and U.S. Pat. Nos. 4,235,871, 4,501,728, 4,837,028, 5,019,369, and 5,260,065, the entire disclosures of which are herein incorporated by reference.

The liposomes encapsulating the thyroid hormone conversion inhibitor can also comprise a ligand molecule that targets the liposome to target cell, such as a skin cancer cell. Ligands which bind to receptors prevalent in such cancer cells, such as monoclonal antibodies that bind to tumor cell antigens or cell surface markers, are preferred.

The liposomes can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both opsonization-inhibition moieties and a ligand. Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the MMS and RES; e.g., as described in U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH.sub.3 and a solvent mixture such as tetrahydrofuran and water in a 30:12 ratio at 60.degree. C.

In certain instances, primarily the stearate derivatives, a sterol such as cholesterol is a particularly useful additive. The addition of cholesterol appears to make the vesicle population more uniform in terms of size and shape. Even cholesterol is not sufficient, in itself, to allow vesicle formation. This is contrast to the materials described in U.S. Pat. No. 4,917,951 which only require cholesterol to make vesicles. In certain circumstances, cholesterol will allow these materials which will not otherwise form a lamellar phase to form a lamellar phase but they cannot be formed into vesicles without the addition of the secondary lipid. In fact, some of the most preferred secondary lipids, e.g., dimethyldistearyl amine, water soluble polyoxyethylene acyl alcohols, and acyl sarcosinate salts, will not form vesicles or lamellar phases either.

In one embodiment of the invention, the locally administrable topical composition is provided for the prevention or treatment of hyperproliferative skin disorders. The locally administrable topical composition includes a topical carrier.

The topical carrier, as noted above, is one which is generally suited to topical drug administration and includes any such materials known in the art. The topical carrier is selected so as to provide the composition in the desired form, e.g., as a liquid, lotion, cream, paste, gel, powder, or ointment, and may be comprised of a material of either naturally occurring or synthetic origin. It is essential that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. The composition of the invention may also be administered in the form of a shampoo, in which case conventional components of such a formulation are included as well, e.g., surfactants, conditioners, viscosity modifying agents, humectants, and the like.

Particularly preferred formulations herein are colorless, odorless ointments, lotions, creams and gels.

Ointments are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (OW) emulsions, and include, for example, cetyl alcohol, glyceryl monostearate, lanolin and stearic acid. Preferred water-soluble ointment bases are prepared from polyethylene glycols of varying molecular weight (Remington: The Science and Practice of Pharmacy).

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose or sodium carboxymethyl-cellulose, or the like. A particularly preferred lotion formulation for use in conjunction with the present invention contains propylene glycol mixed with a hydrophilic petrolatum such as that which may be obtained under the trademark Aquaphor.RTM. from Beiersdorf, Inc. (Norwalk, Conn.).

Creams containing the selected agent are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra, is generally a nonionic, anionic, cationic or amphoteric surfactant.

Gels formulations are preferred for application to the scalp. As will be appreciated by those working in the field of topical drug formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Shampoos may be formulated with the standard shampoo components, i.e., cleansing agents, thickening agents, and preservatives with the cleansing agent representing the primary ingredient, typically an anionic surfactant or a mixture of an anionic and an amphoteric surfactant.

Various additives, known to those skilled in the art, may be included in the topical formulations of the invention. For example, solvents may be used to solubilize certain drug substances. Other optional additives include skin permeation enhancers, opacifiers, anti-oxidants, gelling agents, thickening agents, stabilizers, and the like. Other agents may also be added, such as antimicrobial agents, antifungal agents, antibiotics and anti-inflammatory agents such as steroids.

In preferred topical formulations of the invention, the active agent is present in an amount which is generally at least 1% by weight of the total composition, it can typically range from 0.1 to 40%, for example at least 5%, 10%, etc. Ranges can be for example 1-30%, 5-25%, 10-20% and all other variations are included.

In an alternative embodiment, depending on the disorder to be treated, other amounts of the active agent can be used.

The topical compositions of the invention may also be delivered to the skin using a time-release mechanism. For example, “transdermal”-type patches, wherein the composition is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. The particular polymeric adhesive selected will depend on the particular drug, vehicle, etc., i.e., the adhesive must be compatible with all components of the drug-containing composition. In an alternative embodiment, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the composition and to any other components of the composition, thus preventing loss of any components through the upper surface of the device. The backing layer may be either occlusive or nonocclusive, depending on whether it is desired that the skin become hydrated during drug delivery. The backing is preferably made of a sheet or film of a preferably flexible elastomeric material. Examples of polymers that are suitable for the backing layer include polyethylene, polypropylene, polyesters, and the like.

During storage and prior to use, the laminated structure preferably includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Such devices may be fabricated using conventional techniques, known in the art, for example by casting a fluid admixture of adhesive, drug and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/vehicle mixture.

As with the topical formulations of the invention, compositions contained within the reservoirs of these laminated system may contain a number of components. In some cases, the blocking composition may be delivered “neat,” i.e., in the absence of additional liquid. In most cases, however, the composition will be dissolved, dispersed or suspended in a suitable pharmaceutically acceptable vehicle, typically a solvent or gel. Other components which may be present include preservatives, stabilizers, surfactants, and the like.

Preferably, the topical formulations and the laminated delivery systems also contain a skin permeation enhancer. A skin permeation enhancer can be co-administered. Suitable enhancers are well know in the art and include, for example, dimethylsulfoxide (DMSO), dimethyl formamide (DMF), N,N-dimethylacetamide (DMA), decylmethylsulfoxide (C₁₀MSO), C₂-C₆ alkanediols, and the 1-substituted azacycloheptan-2-ones, particularly 1-n-dodecylcyclazacycloheptan-2-one (available under the trademark Azone.RTM. from Whitby Research Incorporated, Richmond, Va.), alcohols, and the like.

The ointments, pastes, creams and gels also may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays also can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The topical compositions according to this invention may also include one or more preservatives or bacteriostatic agents, e.g., methyl hydroxybenzoate, propyl hydroxybenzoate, chlorocresol, benzalkonium chlorides, and the like. The topical compositions also can contain other active ingredients such as antimicrobial agents, particularly antibiotics, anesthetics, analgesics, and antipruritic agents.

Antimicrobial agents that can be used in the topical administration of the present invention are those compatible with skin and soluble in solvents. In addition, antimicrobial agents are active against a broad spectrum of microorganisms, including but are not limited to, gram positive and gram negative bacteria, yeast, and mold. Examples of the antimicrobial agents include, but are not limited to, triclosan (5-chloro-2-(2,4-dichlorophenoxy)phenol which is also known as Irgasan.TM. DP 300 manufactured by Ciba-Geigy Corporation), hexetidine (5-amino-1,3-bis(2-ethylhexyl)-5-methyl-hexahydropyrimidine), chlorhexidine salts (salts of N,N″-Bis(4-chlorophenyl)-3,12-diimino-2,4,11,14-tetraazatetradecanediimidi amide), 2-bromo-2-nitropropane-1,3-diol, hexyresorcinol, benzalkonium chloride, cetylpyridinium chloride, alkylbenzyldimethylammonium chlorides, iodine, phenol derivatives, povidone-iodine (polyvinylpyrrolidinone-iodine), parabens, hydantoins (2,4-imidazolidinedione), hydantoins derivatives (derivatives of 2,4-imidazolidinedione), phenoxyethanol, cis isomer of 1-(3-chloroallyl)-3,5,6-triaza-1-azoniaadamantane chloride (quarternium-15 which is also known as Dowicil 200 manufactured by Dow Chemical Company), diazolidinyl urea, benzethonium chloride, methylbenzethonium chloride, and mixtures thereof. Examples of hyndantoin derivatives include, but are not limited to, dimethylol-5,5-dimethylhydantoin (glydant). Preferably, examples of the antimicrobial agents include triclosan, cis isomer of 1-(3-chloroallyl)-3,5,6-triaza-1-azoniaadamantane chloride (quarternnium-15), hyndantoins, hyndantoin derivatives such as dimethylol-5,5-dimethylhydantoin (glydant), and mixtures thereof.

Compositions of the present invention can also be useful in conjunction with chemotherapeutic agents in the treatment of skin cancer. Examples of chemotherapeutic agents for the treatment of skin cancer and other hyperproliferative skin disorders are well known in the art.

The topical compositions and drug delivery systems of the invention can be used in the prevention or treatment of the skin conditions identified above. When used in a preventive (prophylactic) method, susceptible skin can be treated prior to exposure or just after exposure but any visible lesions on areas known to be susceptible to such lesions are observed. In treating skin conditions, it will be recognized by those skilled in the art that the optimal quantity and spacing of individual dosages will be determined by the nature and extent of the condition being treated, the form, route and site of administration, and the particular individual undergoing treatment, and that such optimums can be determined by conventional techniques. It will also be appreciated by one skilled in the art that the optimal dosing regimen, i.e., the number of doses can be ascertained using conventional course of treatment determination tests. Generally, a dosing regimen will involve administration of the selected topical formulation at least once daily, and preferably one to four times daily, until the symptoms have subsided.

Although topical administration is preferred, systemic administration of a composition may be by oral, parenteral, sublingual, rectal such as suppository or enteral administration, or by pulmonary absorption. Dosages will be adjusted depending upon how the drug is administered. Parenteral administration may be by intravenous injection, subcutaneous injection, intramuscular injection, intra-arterial injection, intrathecal injection, intra peritoneal injection or direct injection or other administration to one or more specific sites. When long term administration by injection is necessary, venous access devices such as medi-ports, in-dwelling catheters, or automatic pumping mechanisms are also preferred wherein direct and immediate access is provided to the arteries in and around the heart and other major organs and organ systems.

Compositions may also be administered to the nasal passages as a spray. Arteries of the nasal area provide a rapid and efficient access to the bloodstream and immediate access to the pulmonary system. Access to the gastrointestinal tract, which can also rapidly introduce substances to the blood stream, can be gained using oral enema, or injectable forms of administration. Compositions may be administered as a bolus injection or spray, or administered sequentially over time (episodically) such as every two, four, six or eight hours, every day (QD) or every other day (QOD), or over longer periods of time such as weeks to months. Compositions may also be administered in a timed-release fashion such as by using slow-release resins and other timed or delayed release materials and devices.

Where systemic administration is desired, orally active compositions are preferred as oral administration is a convenient and economical mode of drug delivery. Oral compositions may be poorly absorbed through the gastrointestinal lining. Compounds which are poorly absorbed tend to be highly polar. Preferably, such compositions are designed to reduce or eliminate their polarity. This can be accomplished by known means such as formulating the oral composition with a complimentary reagent which neutralizes its polarity, or by modifying the compound with a neutralizing chemical group. Preferably, the molecular structure is similarly modified to withstand very low pH conditions and resist the enzymes of the gastric mucosa such as by neutralizing an ionic group, by covalently bonding an ionic interaction, or by stabilizing or removing a disulfide bond or other relatively labile bond.

Treatments to the patient may be therapeutic or prophylactic. Therapeutic treatment involves administration of one or more compositions of the invention to a patient suffering from one or more symptoms of the disorder. Relief and even partial relief from one or more symptoms can correspond to an increased life span or simply an increased quality of life. Further, treatments that alleviate a pathological symptom can allow for other treatments to be administered.

The term “compatible”, as used herein, means that the components of the compositions are capable of being commingled with the thyroid hormone conversion inhibitors of the present invention, and with each other, in a manner such that does not substantially impair the desired efficacy.

Doses of the pharmaceutical compositions of the invention will vary depending on the subject and upon the particular route of administration used. Dosages can range from 0.1 to 100,000 μg/kg per day, more preferably 1 to 10,000 μg/kg. By way of an example only, an overall dose range of from about, for example, 1 microgram to about 300 micrograms might be used for human use. This dose can be delivered at periodic intervals based upon the composition.

The invention will be further characterized by the following examples which are intended to be exemplary of the invention.

### EXAMPLE

In vitro, T₃ stimulates epidermal keratinocyte proliferation (Holt 1978, Ahsan 1998, Safer 2003). T₃ stimulates cultured keratinocyte expression of proliferation associated keratin gene mRNA (Safer 2004) and protein expression of proliferation associated keratin 6 (Safer, 2005). T₃ also stimulates cultured dermal fibroblast proliferation (Ahsan 1998, Safer 2003).

In vivo, topical application of supra-physiologic T₃ can stimulate epidermal proliferation (Safer 2001, Safer 2003) and may thicken dermis (Faergemann, Yazdanparast).

The predominant circulating thyroid hormone is the pro-hormone, T₄, while thyroid hormone action is mediated by the active thyroid hormone, T₃. Most intracellular T₃ derives from local conversion of T₄ by peripherally expressed iodothyronine deiodinases (DIs). Previous investigators showed conversion of T₄ to either T₃ or inactive rT₃ in skin cultures, thus demonstrating indirectly the presence of thyroid hormone deiodinases in skin (Refetoff 1972, Huang 1985, Kaplan 1988). In vitro, the deiodinase inhibitor, iopanoic acid (IOP), has been used to block T₄ to T₃ conversion in human epidermal keratinocytes (Kaplan—)

Previously, we determined that thyroid hormone is necessary for optimal wound healing (Safer 2004). Thyroid hormone may act on wound healing through dermis, epidermis, or both.

The current project was done to determine whether hypothyroidism would specifically result in decreased epidermal proliferation and whether the topical application of a deiodinase inhibitor, IOP, could inhibit epidermal proliferation without causing systemic hypothyroidism.

Materials and Methods

All animal experimentation described was conducted in accord with accepted standards of humane animal care in a protocol approved by the institutional animal care and use committee (IACUC) at Boston University School of Medicine.

Establishment of Hypothyroid Mice.

Age, sex, and size matched CD-1 mice (Charles River, Boston, Mass.) were thyroidectomized (by surgical thyroidectomy). In order to ascertain hypothyroidism, all mice were eye bled and T₄ levels measured by radio-immunoassay (see below). As with all the murine studies, control mice were subject to the anesthesia, shaving, eye bleeding, and histological analysis used for the treatment animals.

Iopanoic Acid Protocol

A topical iopanoic acid (IOP) cream was prepared by mixing IOP (Sigma, St. Louis, Mo.) into a liposome vehicle (Novasome A, IGI inc., New Jersey) previously described (Safer 2001, Safer 2003, Safer 2005). Each mouse received 30 μl of the liposome vehicle applied directly to a 3×3 cm area of shaved skin on the animal's backs. The preparation was applied daily and not removed.

The treatment groups received cream containing 6 mg IOP daily. The control groups were treated with daily application of vehicle alone.

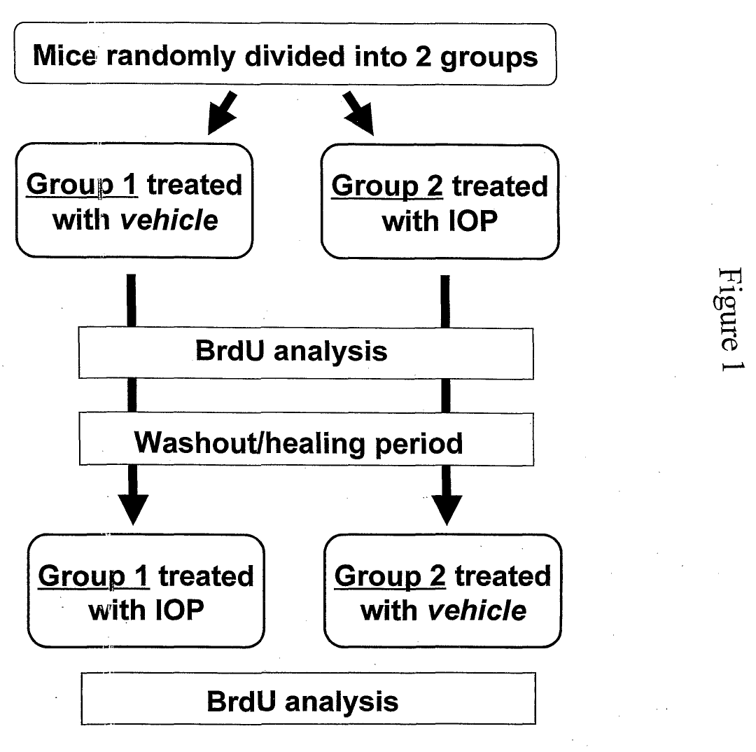

Five week old CD-1 mice were randomized into 2 groups (FIG. 1). The groups had 6 mice each and were evaluated in a crossover study. Each mouse in the crossover study was evaluated twice: once after a 6 mg daily IOP treatment and once after treatment with vehicle alone. Six mice received IOP first followed by a 3 month wash-out/healing completion period. The animals were then evaluated after treatment with topical vehicle alone. Six mice started with vehicle alone and received the IOP treatment after the 3 month wash-out/healing completion period.

All topically treated mice were caged separately to avoid animals receiving doses from their neighbors during grooming. The cream was applied to a midline cephalad region on the animals' backs to minimize the impact of personal grooming on dosing.

Measurement of Serum T₄ Levels

Serum total thyroxine levels were measured with a standard radioimmunoassay kit (ICN, Orangeburg, N.Y.). Unlike other thyroid hormone kits which use anti-mouse antibodies, the ICN kit uses anti-rabbit antibodies and avoids spuriously elevated readings in mice. Thyroid hormone levels for mice fall at the low end of the human range so the human standards included in the kit were used.

Evaluation of Epidermal Proliferation

Epidermal proliferation was assessed by measuring both epidermal thickness and 5-bromo-2′-deoxyuridine (BrdU, Boehringer Mannheim, Mannheim, Germany) incorporation into DNA. At the completion of the protocols noted, animals were biopsied with full thickness dorsal skin samples were taken. Three hours prior to biopsy, each mouse had received intraperitoneal BrdU as previously described (Safer 2001). Skin samples were fixed in formalin and embedded in paraffin. From each paraffin block, 5 Mm sections were made and stained with hematoxylin and eosin (H&E). Epidermal thickness was measured in 10 random locations for each mouse. Additional 5 μm skin histology sections from the above paraffin blocks were stained for BrdU as previously described (Safer 2001). BrdU data are reported as the number of cell nuclei stained/nm epidermis.

Human Epidermal Keratinocyte Studies

Human keratinocytes were grown in primary culture on a support matrix derived from irradiated fibroblasts. When the cultures reached 40% confluence, the cells were fed basal medium alone for 24 hours and then incubated overnight in medium containing a test substance or sham. The cells were then treated with saturating quantities of ³H-thymidine (New England Nuclear, Boston, Mass.); DNA was precipitated with 5% perchlorate and relative incorporated thymidine was assessed with a beta counter. Experiments were performed in quadruplicate and averaged. Results reported represent a minimum of three separate quadruplicate experiments.

The skin discards were obtained anonymously following accepted procedures set by the Institutional Review Board at Boston University School of Medicine.

Human Prostate Cell Culture Studies

Human prostate (HPV) cells were grown under standard conditions. When the cultures reached 40% confluence, the cells were fed basal medium alone for 24 hours and then incubated overnight in medium containing a test substance or sham. The cells were then treated with saturating quantities of ³H-thymidine (New England Nuclear, Boston, Mass.); DNA was precipitated with 5% perchlorate and relative incorporated thymidine was assessed with a beta counter. Experiments were performed in quadruplicate and averaged. Results reported represent a minimum of three separate quadruplicate experiments.

Opossum Kidney Cell Culture Studies

Opossum kidney (OK) cells were grown under standard conditions. When the cultures reached 40% confluence, the cells were fed basal medium alone for 24 hours and then incubated overnight in medium containing a test substance or sham. The cells were then treated with saturating quantities of ³H-thymidine (New England Nuclear, Boston, Mass.); DNA was precipitated with 5% perchlorate and relative incorporated thymidine was assessed with a beta counter. Experiments were performed in quadruplicate and averaged. Results reported represent a minimum of three separate quadruplicate experiments.

Monkey Kidney Cell Culture Studies

Monkey kidney (CV-1) cells were grown under standard conditions. When the cultures reached 40% confluence, the cells were fed basal medium alone for 24 hours and then incubated overnight in medium containing a test substance or sham. The cells were then treated with saturating quantities of ³H-thymidine (New England Nuclear, Boston, Mass.); DNA was precipitated with 5% perchlorate and relative incorporated thymidine was assessed with a beta counter. Experiments were performed in quadruplicate and averaged. Results reported represent a minimum of three separate quadruplicate experiments.

Reagents

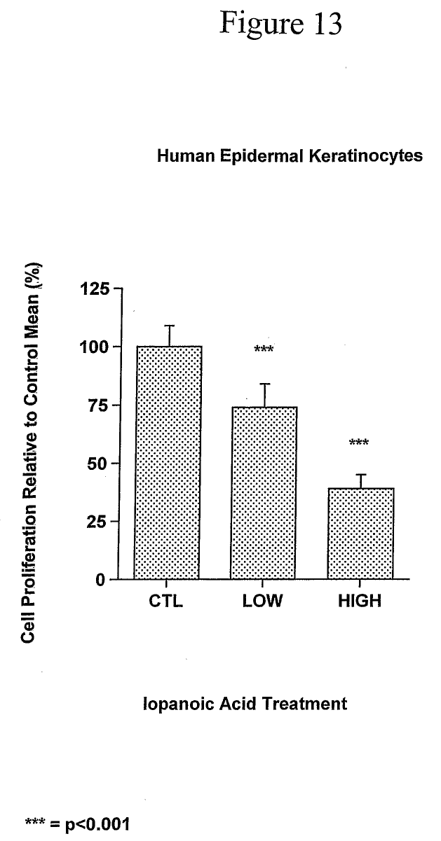

The reagents used for tissue culture were iopanoic acid (Sigma, St. Louis, Mo.), sodium ipodate (Fitzgerald Industries International, Flanders, N.J.), and propranolol (Sigma, St. Louis, Mo.). Reagents were dissolved into modestly basic stock solutions to the following final concentrations: 0.025 mM NaOH, 0.2 M NaCl, 1.5 mM reagent. Each experiment used 10 μl of stock reagent (or sham) per 500 μl of medium. For FIGS. 13-18, the “low” concentration is 3 μM for each reagent, and the “high” concentration was 30 μM for each reagent.

Statistics

Statistical analysis was performed with Student's unpaired t test. Data is presented ±standard error of the mean (SEM).

Results

Topical IOP Inhibited Epidermal Proliferation

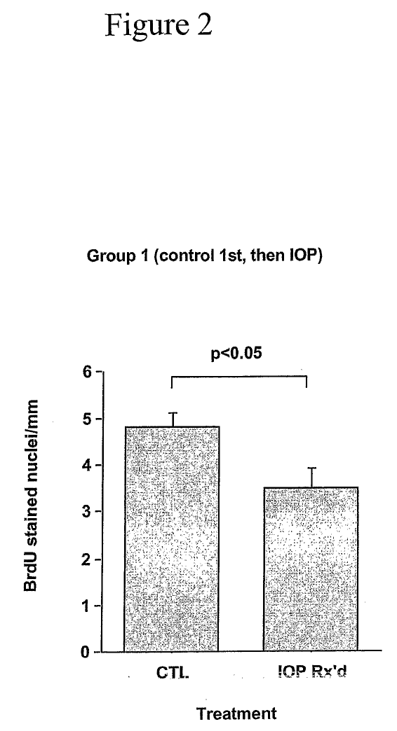

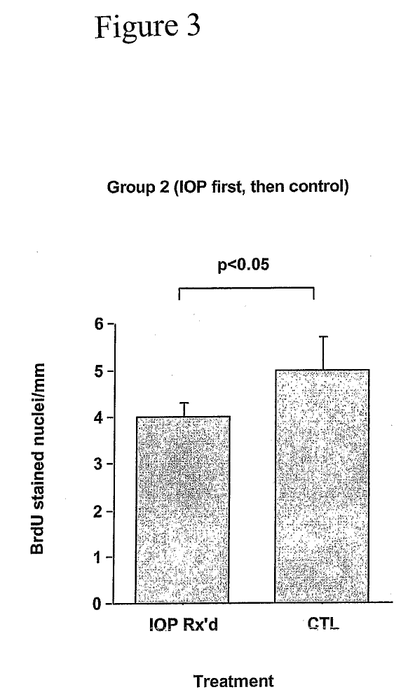

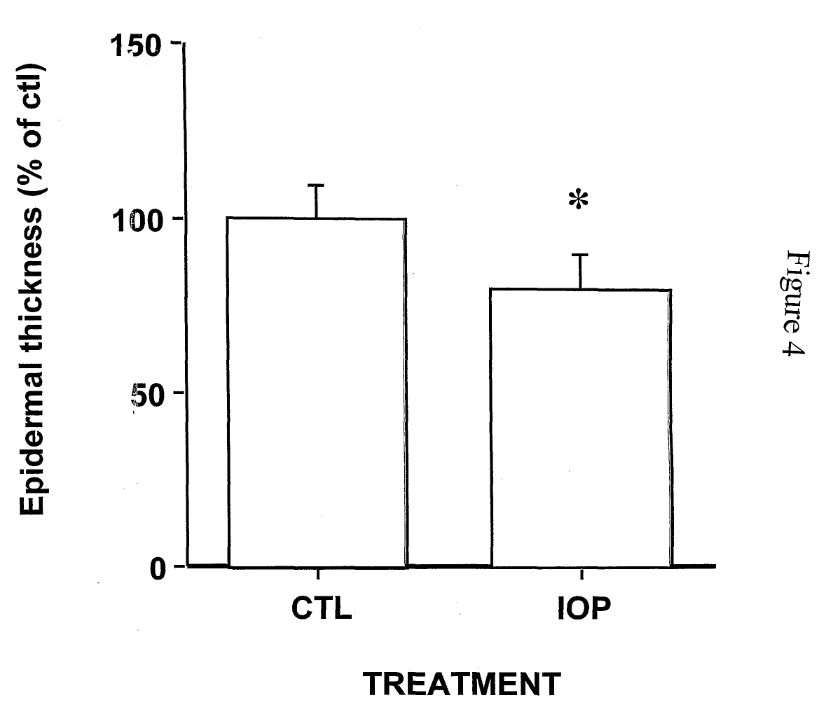

Epidermal proliferation was significantly diminished in both groups of mice (FIGS. 2 and 3). Thyroidectomized mice had 36% thinner epidermis than control mice (p<0.05) and 90% less epidermal BrdU staining than controls (p<0.05). Mice treated with topical IOP had 20% thinner epidermis than control mice (p<0.05; FIG. 4 and 30% less epidermal BrdU staining (p<0.05). While serum T₄ levels fell significantly in the thyroidectomized mice, T₄ levels in mice treated with IOP cream remained steady.

T₄ Levels were Diminished in Thyroidectomized Mice.

Relative to baseline, T₄ levels in thyroidectomized mice were 84% lower (4.2±0.4 μg/dl for euthyroid mice versus 0.66±0.5 μg/dl for hypothyroid animals, p<0.001). By contrast, T₄ levels in IOP treated mice were not changed relative to control mice.

Deiodinase Inhibitors Had Effects on Multiple Tissue Samples from Different Species.

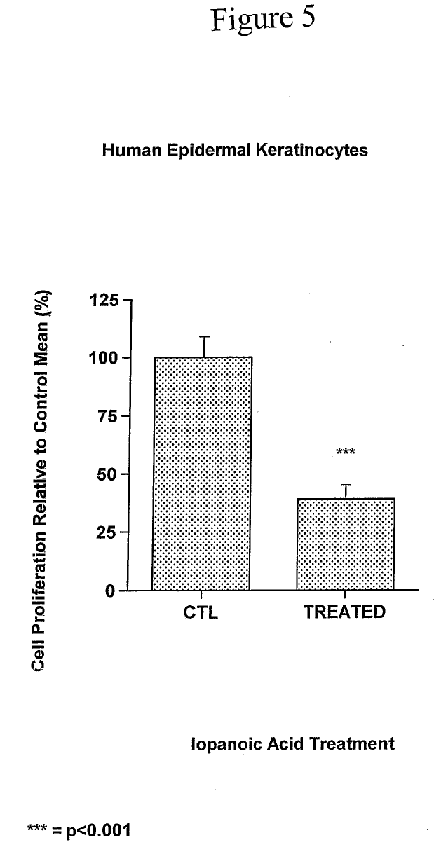

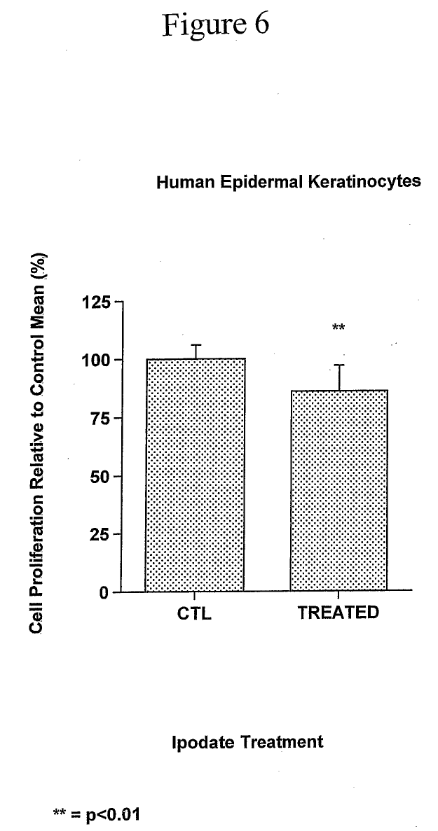

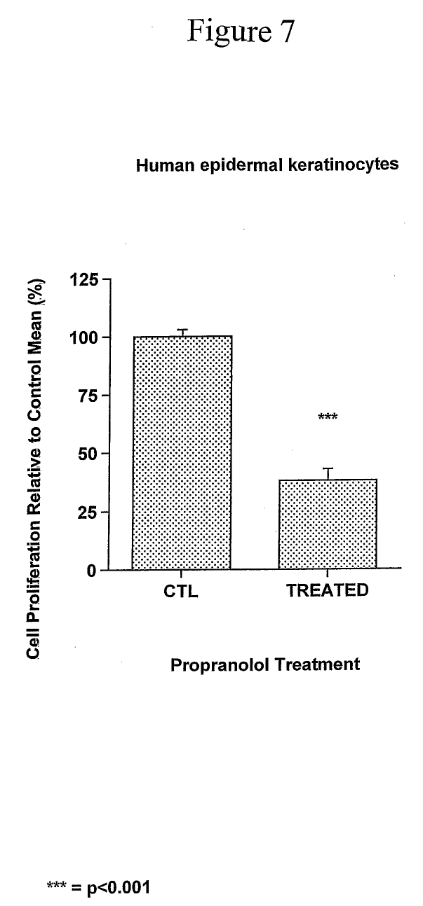

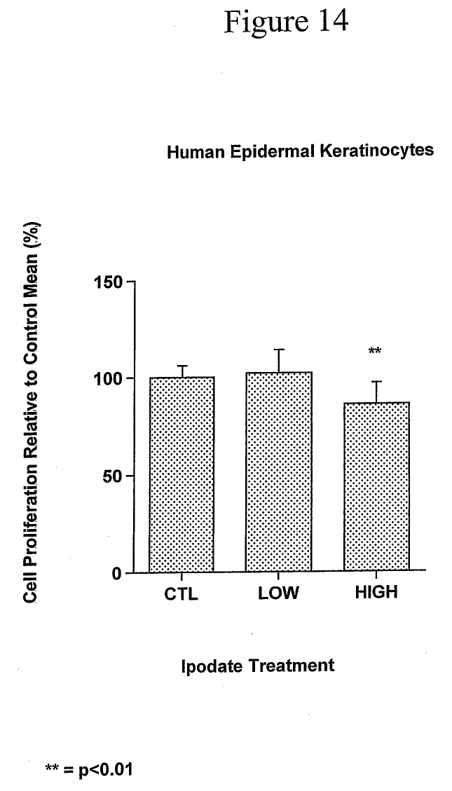

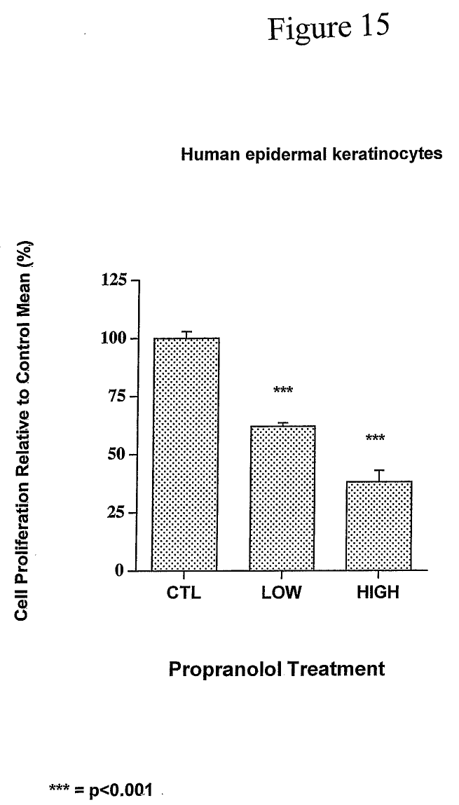

As shown in FIG. 5, relative to control cultures, proliferation of human epidermal keratinocytes was inhibited 61±9% (p<0.001) after incubation overnight with iopanoic acid. Similarly, FIG. 13 shows the results of the human epidermal keratinocytes incubated with two different concentrations of iopanoic acid. As shown in FIG. 6, proliferation of human epidermal keratinocytes was inhibited 14±6% (p<0.01) after incubation overnight with ipodate sodium; FIG. 14 shows the results of the human epidermal keratinocytes incubated with two different concentrations of ipodate sodium. FIG. 7 shows that proliferation of keratinocytes was inhibited 62±3% (p<0.001) after overnight incubation with propranolol. Again, FIG. 15 shows the results of the human epidermal keratinocytes incubated with two different concentrations of propanolol.

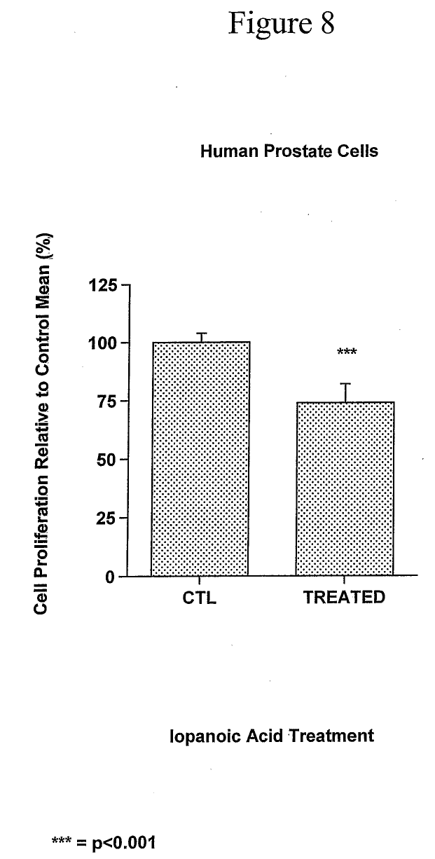

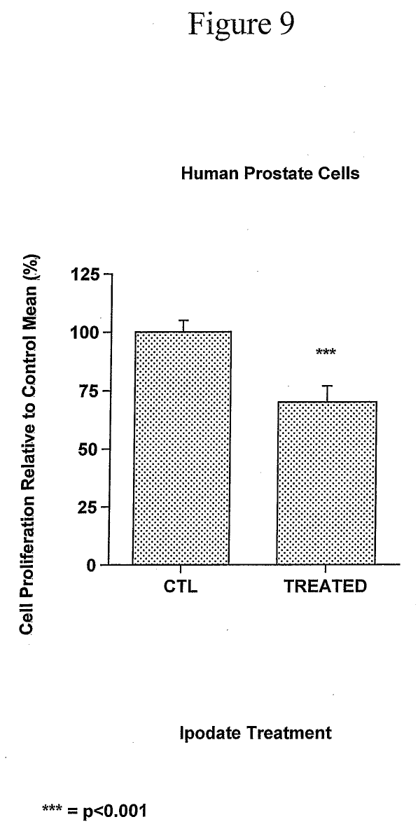

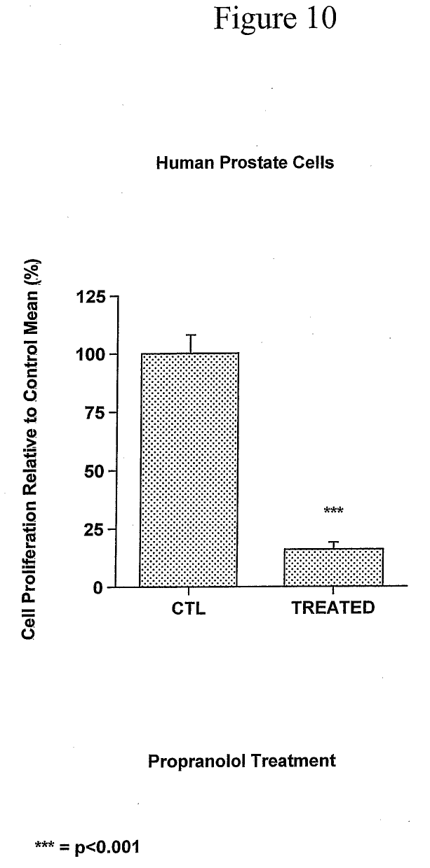

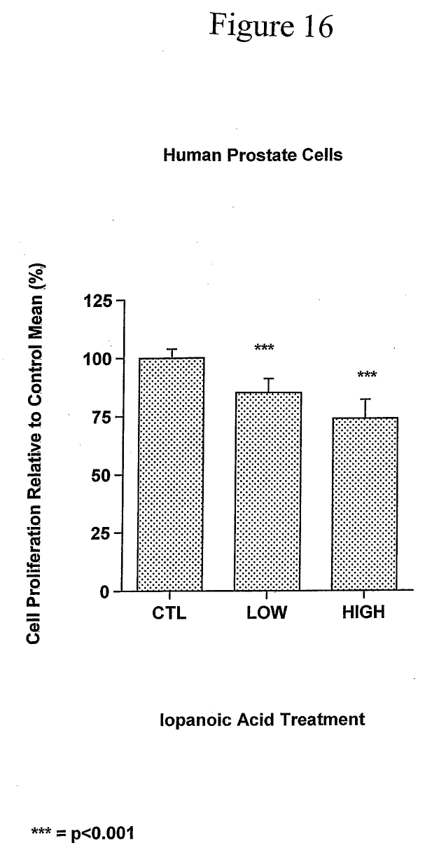

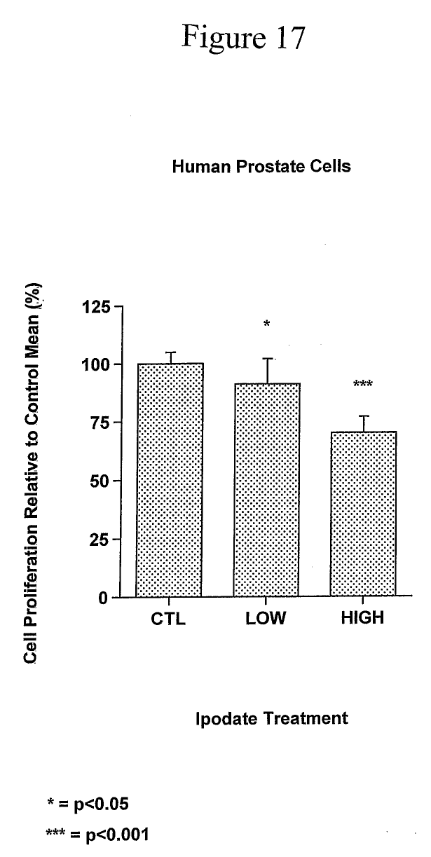

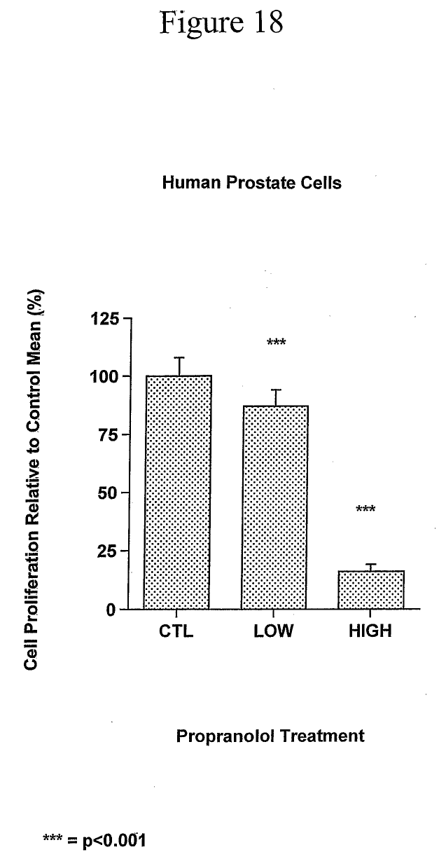

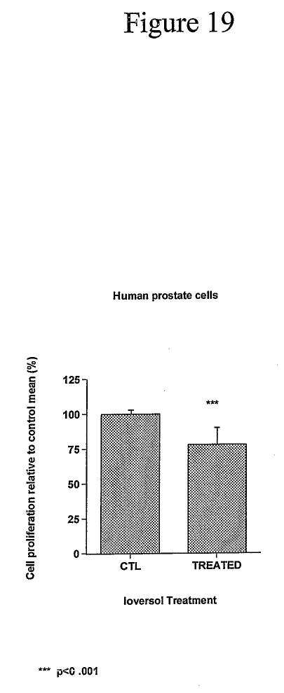

FIG. 8 shows that relative to control cultures, proliferation of human prostate cells was inhibited 26±4% (p<0.001) after incubation overnight with iopanoic acid. FIG. 16 shows the results of the human prostate cells incubated with two different concentrations of iopanoic acid. In FIG. 9, proliferation of human prostate cells was inhibited 30±5% (p<0.001) after incubation overnight with ipodate sodium. Similarly, FIG. 17 shows the results of the human prostate cells incubated with two different concentrations of ipodate sodium. As shown in FIG. 10, proliferation of prostate cells was inhibited 84±8% (p<0.001) after overnight incubation with propranolol. FIG. 18 shows the results of the human prostate cells incubated with two different concentrations of propanolol. In FIG. 19, proliferation of human prostate cells incubated overnight with ioversol was inhibited 22% (+/−7%, p<0.001).

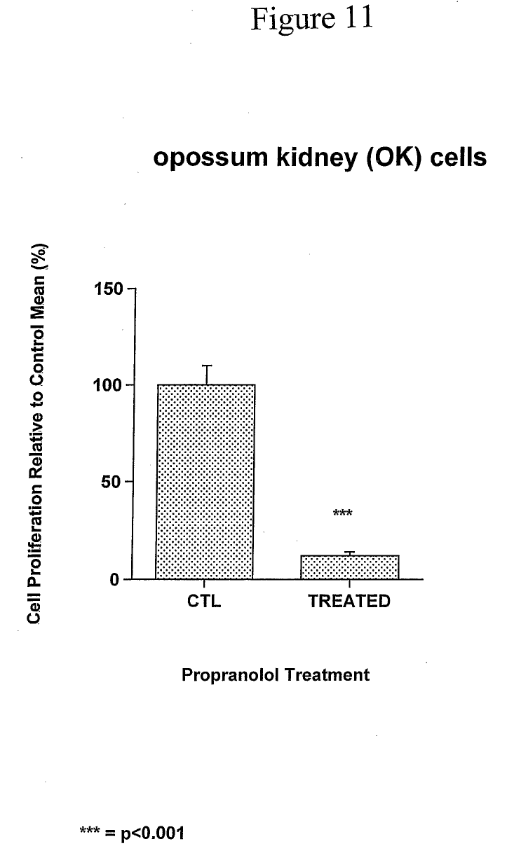

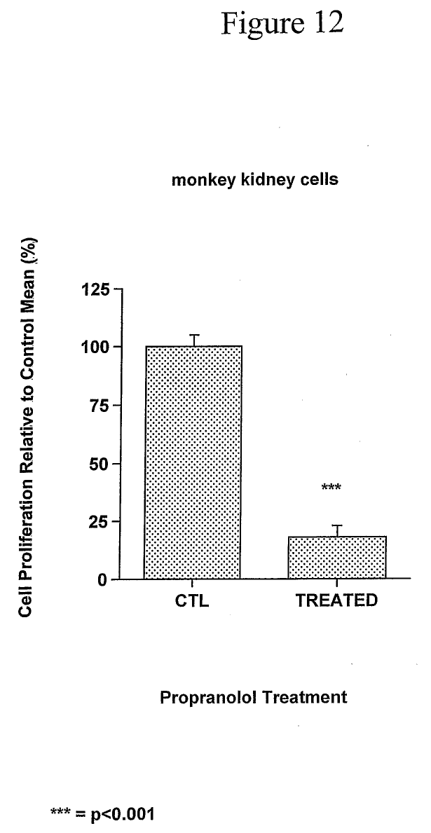

FIG. 11 shows that relative to control cultures, proliferation of opossum kidney cells was inhibited 88±10% (p<0.001) after overnight incubation with propranolol. In FIG. 12, proliferation of monkey kidney cells was inhibited 82±5% (p<0.001) after overnight incubation with propranolol.

### REFERENCES

Ahsan M K, Urano Y, Kato S, Oura H, Arase S 1998 Immunohistochemical localization of thyroid hormone nuclear receptors in human hair follicles and in vitro effect of L-triiodothyronine on cultured cells of hair follicles and skin. Journal of Medical Investigation 44:179-184
Faergemann J, Sarnhult T, Hedner E, Carlsson B, Lavin T, Zhao X-E, Sun X-Y 2002 Dose-response effects of tri-iodothyroacetic acid (Triac) and other thyroid hormone analogues on glucocorticoid-induced skin atrophy in the haired mouse. Acta Derm Venerol 82:179-183
Holt P J A 1978 In vitro responses of the epidermis to triiodothyronine. Journal of Investigative Dermatology 71:202-204
Huang T S, Chopra I J, Beredo A, Solomon D H, Chua Teco G N 1985 Skin is an active site for the inner ring monodeiodination of thyroxine to 3,3′,5′-triiodothyronine. Endocrinology 117:2106-2113
Kaplan M M, Pan C, Gordon P R, Lee J K, Gilchrest B A 1988 Human epidermal keratinocytes in culture convert thyroxine to 3,5,3′-triiodothyronine by type II iodothyronine deiodination: a novel endocrine function of skin. Journal of Clinical Endocrinology and Metabolism 66:815-822
Refetoff S, Matalon R, Bigazzi M 1972 Metabolism of L-thyroxine (T₄) and L-triiodothyronine (T₃) by human fibroblasts in tissue culture: evidence for cellular binding proteins and conversion of T₄ to T₃. Endocrinology 91:934-947
Safer J D, Fraser L M, Ray S, Holick M F 2001 Topical triiodothyronine stimulates epidermal proliferation, dermal thickening, and hair growth in mice and rats. Thyroid 11:717-724
Safer J D, Crawford T M, Fraser L M, Hoa M, Ray S, Chen T C, Persons K, Holick M F 2003 Thyroid hormone action on skin: Diverging effects of topical versus intraperitoneal administration. Thyroid 13:159-165
Safer J D, Crawford T M, Holick M F 2004 A role for thyroid hormone in wound healing through keratin gene expression. Endocrinology 145:2357-2361
Safer J D, Crawford T M, Holick M F 2005 Topical Thyroid Hormone Accelerates Wound Healing in Mice. Endocrinology In Press
Yazdanparast P, Carlsson B, Oikarinen A, Risteli J, Faergemann J 2004 A thyroid hormone analogue, triiodothyroacetic acid, corrects corticosteroid-down-regulated collagen synthesis. Thyroid 14:345-353

All references described herein are incorporated by reference.

We claim:

1. A method for treating a hyperproliferative skin disorder, comprising topically administering to a subject in need thereof an effective amount of a thyroid hormone conversion inhibitor and a pharmaceutically acceptable carrier or diluent.

2. The method of claim 4, wherein the method comprises locally inhibiting the conversion of thyroid pro-hormone, T₄, to thyroid hormone, T₃.

3. The method of claim 1, wherein the thyroid hormone conversion inhibitor is selected from the group consisting of iopanoic acid (IOP), ipodate, and propranolol.

4. The method of claim 3, wherein the thyroid hormone conversion inhibitor is a deiodinase inhibitor.

5. The method of claim 1, wherein the hyperproliferative skin disorder is selected from the group consisting of psoriasis, acne vulgaris, acne rosacea, actinic keratosis, solar keratoses, squamous carcinoma in situ, the ichthyoses, hyperkeratoses, disorders of keratinization such as Darriers disease, palmoplanter keratodermas, pityriasis rubra pilaris, epidermal naevoid syndromes, erythrokeratoderma variabilis, epidermolytic hyperkeratoses, non-bullous ichthyosiform erythroderma, cutaneous lupus erythematosus and lichen planus.psoriasis, acne vulgaris, or cancer.

6. The method of claim 5, wherein the hyperproliferative skin disorder is psoriasis.

7. The method of claim 5, wherein the hyperproliferative skin disorder is the ichthyoses.

8. The method of claim 5, wherein the hyperproliferative skin disorder is palmoplanter keratodermas.

9. The method of claim 5, wherein the pharmaceutically acceptable carrier or diluent is a liposome cream and the thyroid conversion is for topical administration.

10. The method of claim 5, wherein the endogenous level of thyroid hormone, T₃, is lowered locally in the cells of the subject treated with the inhibitor, and further wherein the subject's systemic level of thyroid hormone is not significantly altered.

11. A pharmaceutical composition for topical administration comprising a sufficient amount of a deiodinase inhibitor to inhibit conversion of T₄ to T₃, wherein thyroid hormone conversion inhibitor and a pharmaceutically acceptable carrier or diluent.

12. The pharmaceutical composition of claim 11, wherein the pharmaceutically acceptable carrier or diluent is selected from the group consisting of lotion, cream, paste, gel and ointment.

13. The pharmaceutical composition of claim 1, wherein the pharmaceutically acceptable carrier or diluent is a liposome cream.
